(19) United States
(12) Patent Application Publication
(10) Pub. No.: US 2026/0216286 A1
(43) Pub. Date: Jul. 30, 2026

(54) THERAPEUTIC PROTEIN COMBINATIONS

(71) Applicant: OHIO STATE INNOVATION FOUNDATION, Columbus, OH (US)

(72) Inventors: Andre PALMER, Columbus, OH (US); Katelyn Elizabeth REILLY, Columbus, OH (US); Megan ALLYN, Columbus, OH (US)

(21) Appl. No.: 19/159,002

(22) PCT Filed: Feb. 23, 2024
(86) PCT No.: PCT/US24/17135

§ 371 Date: Aug. 22, 2025

Related U.S. Application Data
(60) Provisional application No. 63/447,767, filed on Feb. 23, 2023.

(51) Int. Cl. A61K 38/17; A61K 38/40

(52) U.S. Cl. CPC A61K 38/1722; A61K 38/1741; A61K 38/40

(57) ABSTRACT

Disclosed herein are compositions including one or more therapeutic proteins. The therapeutic proteins are useful in the treatment of ocular injuries and disorders.

### CROSS-REFERENCE TO RELATED APPLICATION

This application claims the benefit of U.S. Provisional Application 63/447,767, filed Feb. 23, 2023, the contents of which are hereby incorporated in its entirety.

### STATEMENT OF GOVERNMENT SUPPORT

This invention was made with government support under HL162120 awarded by the National Institutes of Health and contract HT94252310782 awarded by the Department of Defense. The government has certain rights in the invention.

### FIELD OF THE INVENTION

The invention relates to therapeutic protein compositions including haptoglobin, hemopexin, transferrin, or a combination thereof. In some implementations the compositions include particles including the therapeutic protein(s). In some implementations the therapeutic protein(s) can be used to treat a variety of injuries, including ocular injuries and traumatic brain injuries.

### BACKGROUND

Approximately 2.4M ocular injuries occur in the US per year, with nearly 35% of injuries impacting younger patients. In the military, ocular injuries comprise between 5-10% of all injuries. However, treatment of ophthalmic injuries is complex and requires specialized care with extensive resources. Furthermore, in many instances military personnel can only return to limited duty due to lingering visual issues. Local treatment shortly after ocular injury in civilian and military combat with reactive oxygen species (ROS) scavenging polydopamine (PDA) nanoparticles (NPs) and protein scavengers such as haptoglobin (Hp), hemopexin (Hpx), and transferrin (Tf) to alleviate hemoglobin (Hb), heme and iron toxicity may prevent vision loss and promote a conducive environment for healing after injury.

Outcomes after ocular injury remain poor, relating strongly to the lack of early intervention and misdiagnosis. Delayed or abstained treatment of retinal detachment and/or retinal pathologies has the potential to devolve into a complication called proliferative vitreoretinopathy (PVR). Damage to the blood-retinal-barrier (BRB) can correlate to increased permeability of cytotoxic blood components into the retinal space, exposing the normally protected outer retinal neurons and retinal pigment epithelial (RPE) cells to excess extracellular iron and heme. Enhanced oxidative stress and inflammation caused by free iron and heme, as a result of saturation of the endogenous adaptive scavenging protein response induces changes such as migration of RPE cells, angiogenesis, and dedifferentiation/mesenchymal transition (MT)of Müller glial cells and retinal pigment epithelial cells, or that leads to fibrotic scarring and vision loss.

Polydopamine nanoparticles have been established as a biocompatible drug delivery vehicle and have shown antioxidant (oxidative radical scavenging) capabilities that provide oxidative stress relief. In this project, these nanoparticles were explored in conjunction with a designed protein therapeutic as a novel strategy to combat dedifferentiation of retinal cells as an in vitro model for exacerbation to PVR in patients after ocular trauma and retinal detachment.

There remains a need for improved systems and methods for delivering therapeutic proteins to tissue systems of interest. There remains a need for improved systems and methods for treating disorders associated with excess iron, including excess intracellular iron. There remains a need for improved systems and methods for treating tissue injuries, including traumatic brain injuries and ocular injuries.

### BRIEF DESCRIPTION OF THE FIGURES

FIG. 1 depicts HPLC-SEC of haptoglobin (Hp) (top); and Hp, hemopexin (Hpx) and transferrin (Tf) protein cocktail (bottom), along with their hemoglobin (Hb) binding activity.

FIG. 2 depicts MTS assay demonstrating no adverse effects on ARPE-19 cell viability after exposure to haptoglobin (Hp), hemopexin (Hpx) and transferrin (Tf) protein cocktail even at 100 μg/mL.

FIG. 3 depicts the preliminary impact of the therapeutic protein cocktail (TPC) containing Hp, Hpx and Tf to reduce oxidative stress in pRPE cells.

FIG. 4 depicts the preliminary impact of TPC to reduce oxidative stress in pMüller cells.

FIG. 5 depicts phenotype verification of pMüller cells by immunofluorescence of glutamate synthetase and glial fibrillary associated protein. Antibody expression was visualized with Alexa Fluor 555.

FIG. 6 depicts the expression of vimentin and S100A4 with and without treatment of TPC. TPC application shows decreased fluorescence of S100A4, indicative of a decrease in fibroblast-like cells when compared to control. No clear change is shown in expression of vimentin.

FIG. 7 depicts oxidative stress reduction by TPC-loaded nanoparticles.

FIG. 8A depicts microscopy images of polydopamine nanoparticles and polydopamine nanoparticles loaded with TPC.

FIG. 8B depicts polydopamine nanoparticles enter retinal cells within 24 hours of incubation.

FIG. 9 depicts that higher concentrations of H₂O₂ led to faster NP degradation and release of FITC-BSA.

FIG. 10 depicts polydopamine nanoparticles exhibiting the ability scavenge and reduce intracellular ROS induced by H₂O₂, as shown by decrease in green DCFH-DA probe with increased PDA NP concentration (n=3, p<0.05). Scale bar=300 μm.

FIG. 11A depicts HPLC-SEC of Hp (top) and Hp/Hpx/Tf protein cocktail (bottom) with their Hb binding activity.

FIG. 11B depicts a MTS assay demonstrating no adverse effects on ARPE-19 cell viability after exposure to TPC even at 10,000 μg/mL.

FIG. 11C depicts Increasing TPC concentration decreased oxidative stress after exposure to H₂O₂ in pRPE cells, indicating reduction of ROS in culture. Results are normalized to positive control of oxidatively challenged cells with no TPC applied.

FIG. 11D depicts Increasing TPC concentration decreased oxidative stress after exposure to H2O₂ in pMüller cells, indicating reduction of ROS in culture. Results are normalized to positive control of oxidatively challenged cells with no TPC applied.

FIG. 12 depicts TPC treatment decreases expression of S100A4, indicative of a decrease in fibroblast-like cells.

FIG. 13 depicts a schematic of the tangential flow filtration system to clarify and concentrate TPC.

FIG. 14A depicts the Stage 4 product in FIG. 13 characterized by SDS-PAGE in native (A) and denatured (B) conditions. (C) Tryptic digest mass spectrometry was also performed to confirm protein composition and is shown to contain significant scavenging proteins.

FIG. 14B depicts characterization of the Stage 4 product in FIG. 13 and FIG. 14A.

FIG. 15 depicts that cytotoxicity of TPC was shown to be negligible by MTS assay at 2 mg/mL. Inclusion of TPC into PDA NPs did not impact the cytotoxicity of 20 ug/mL nanoparticles. Cells were plated in 96 well and treated with therapeutics 24 hours after adherence. Cell viability was measured 24 hours after treatment.

FIG. 16 depicts the ability of TPC and TPC-PDA NPs to combat oxidative stress from reactive oxygen species model (H₂O₂) in primary Müller Glial cells (pMüller) extracted from porcine eyes determined by DCF assay. Cells were seeded at 15 k/well and allowed to adhere.

FIG. 17 depicts the ability of TPC and TPC-PDA NPs to combat oxidative stress from reactive oxygen species model (H₂O₂) in primary retinal pigment epithelial cells (pRPE) extracted from porcine eyes determined by DCF assay. Cells were seeded at 40 k/well and allowed to adhere.

FIG. 18 depicts the ability of TPC to mitigate mitochondrial oxidative stress and mesenchymal transition related proteins in pMüller cells after ROS challenge was investigated via immunofluorescence. Increased fluorescence shows an increase in production that is quelled by TPC.

FIGS. 19A, 19B, and 19C depict the ability of TPC and TPC-PDA to combat oxidative stress from iron toxicity in pRPE and pMüller Glial cells was determined by DCF assay. Cells were seeded at 40 k/well for RPE cells and 15 k/well Muller Glial cells and allowed to adhere.

FIG. 20 depicts the ability of TPC to mitigate mesenchymal transition related proteins in Muller Glial Cells after ROS challenge was investigated via immunofluorescence. Increased fluorescence shows an increase in production that is quelled by TPC.

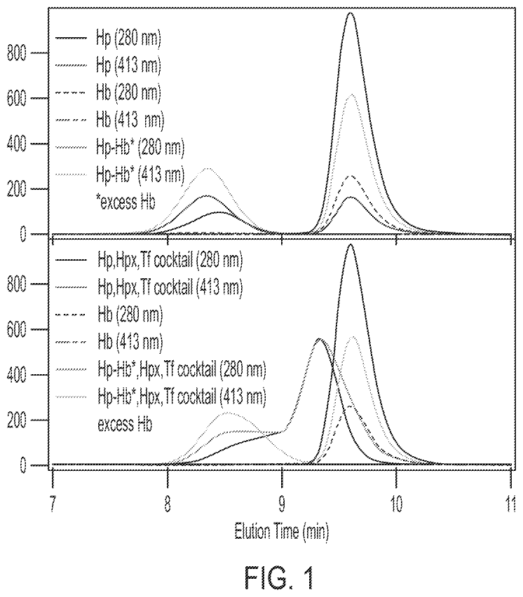

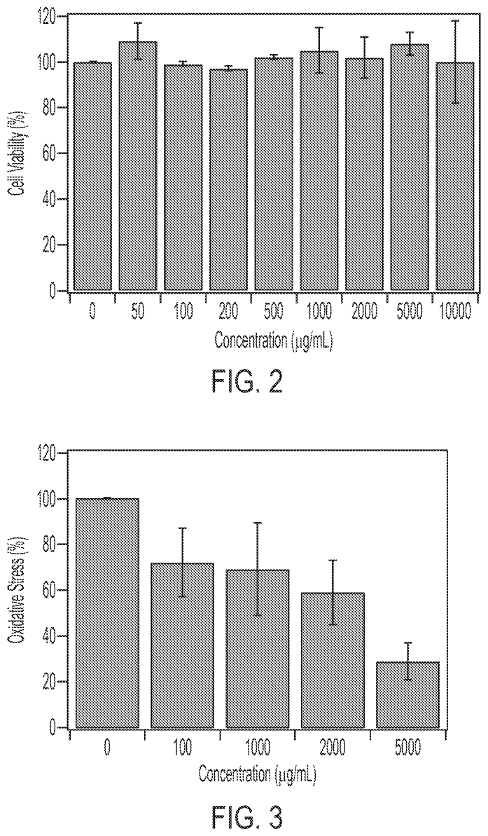

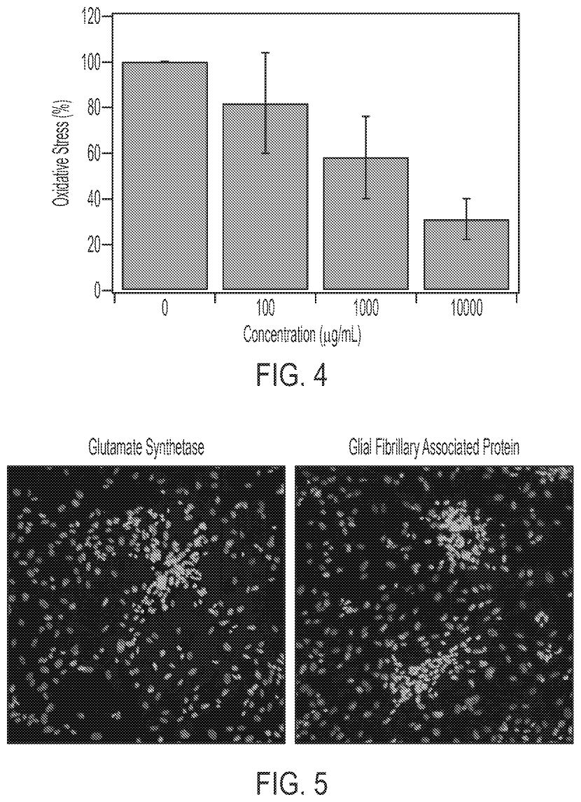

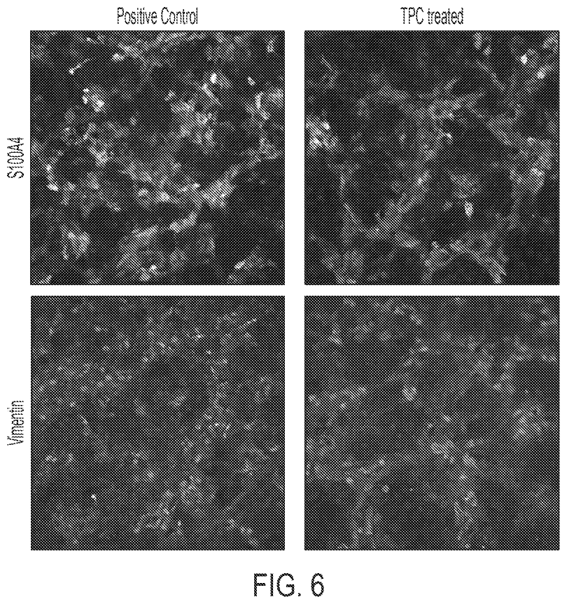

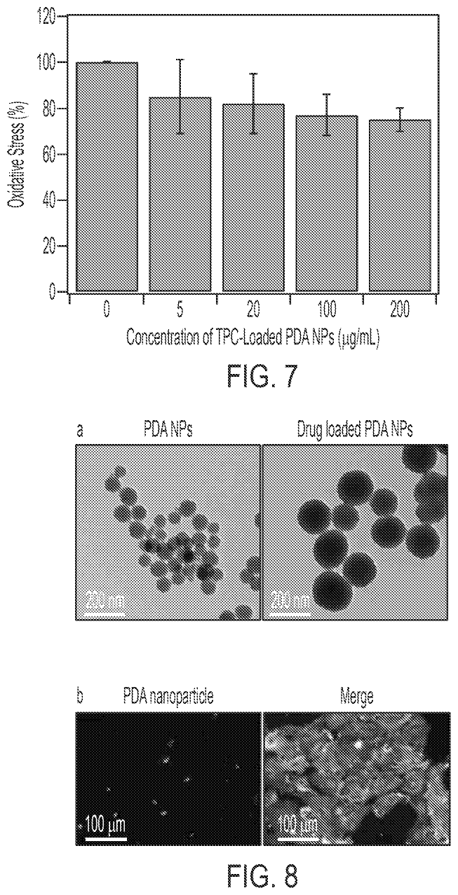

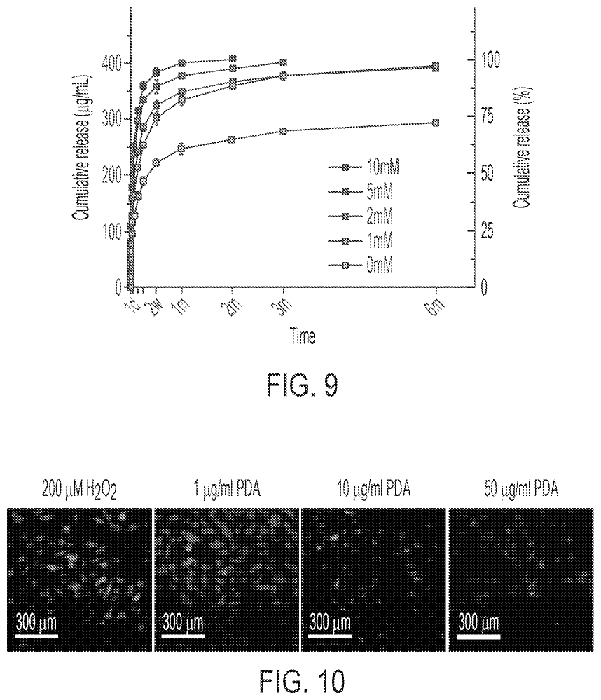

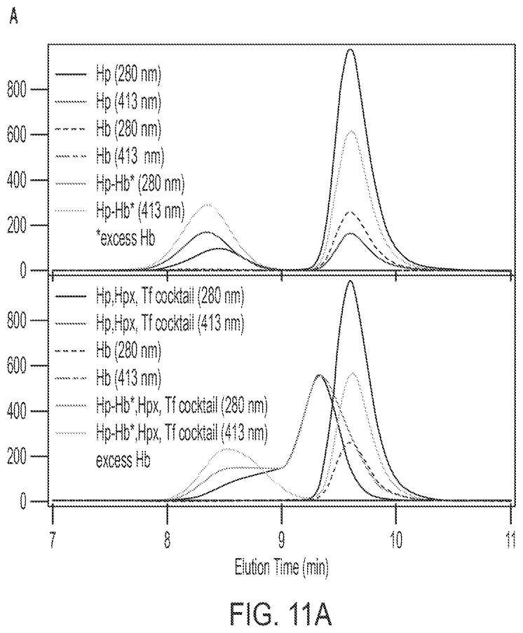

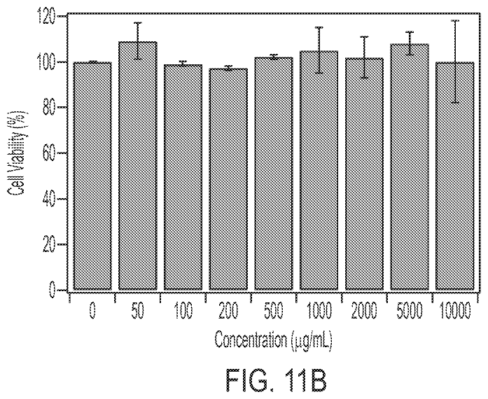

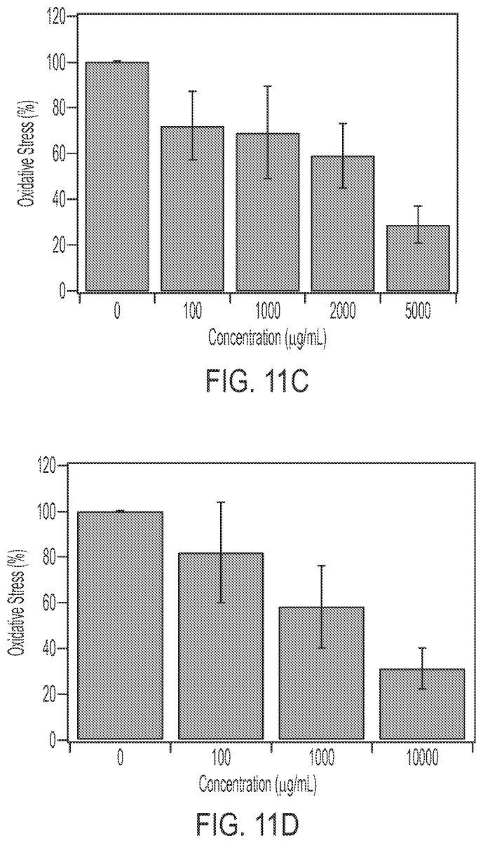

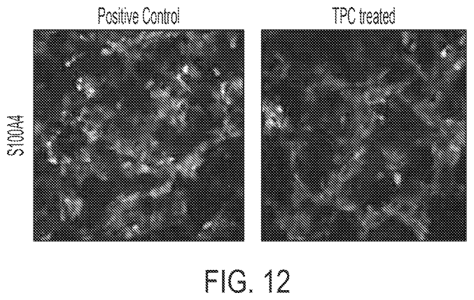

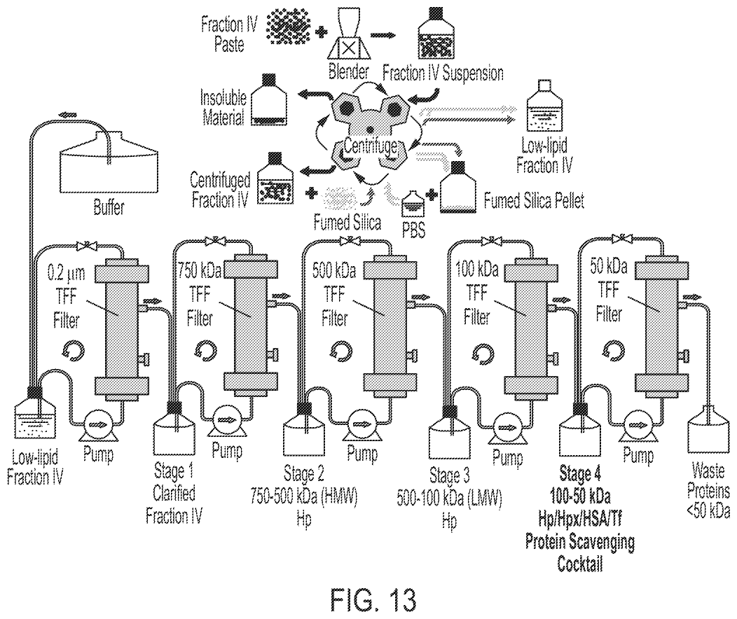

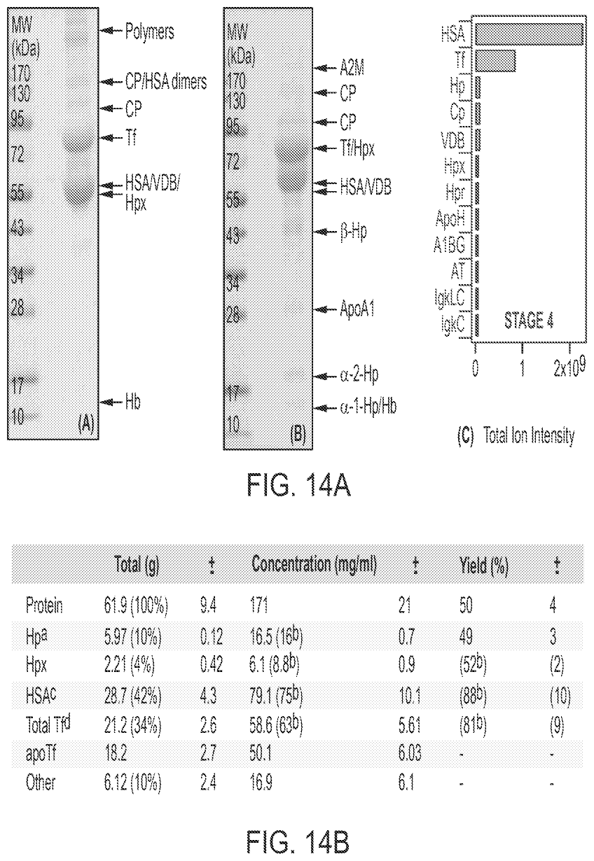

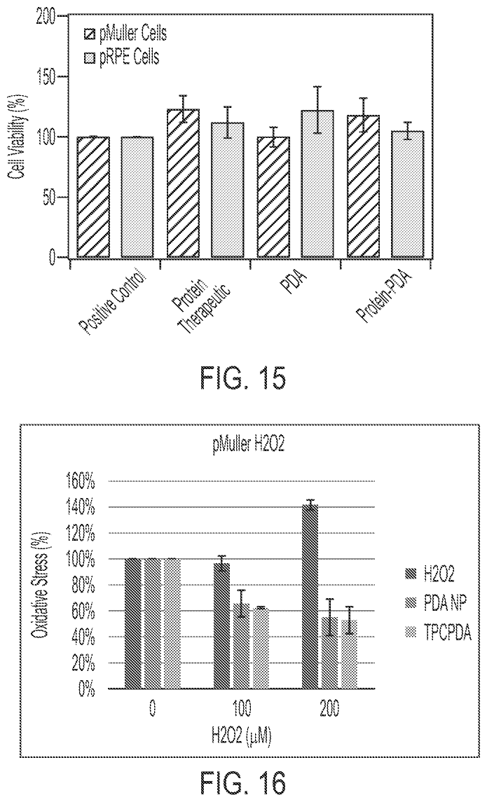

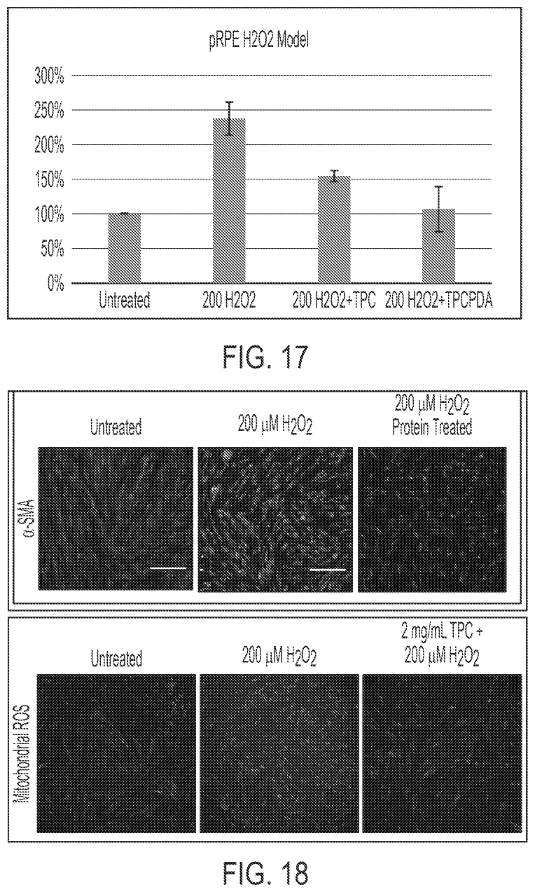

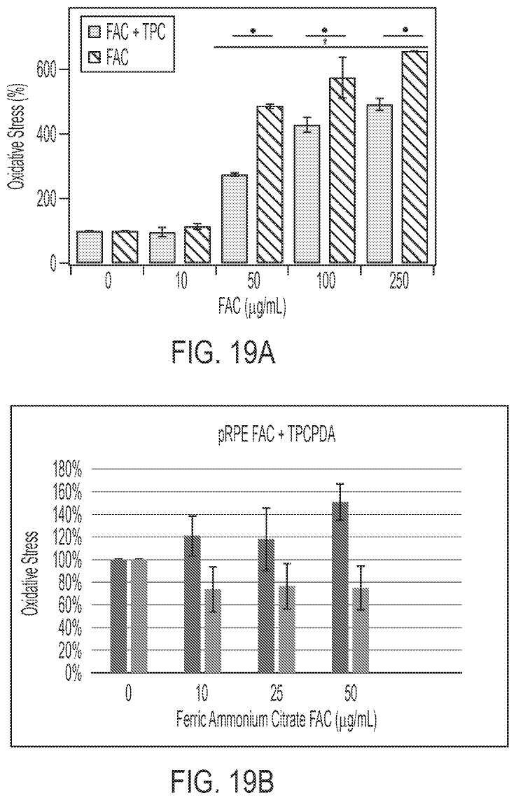

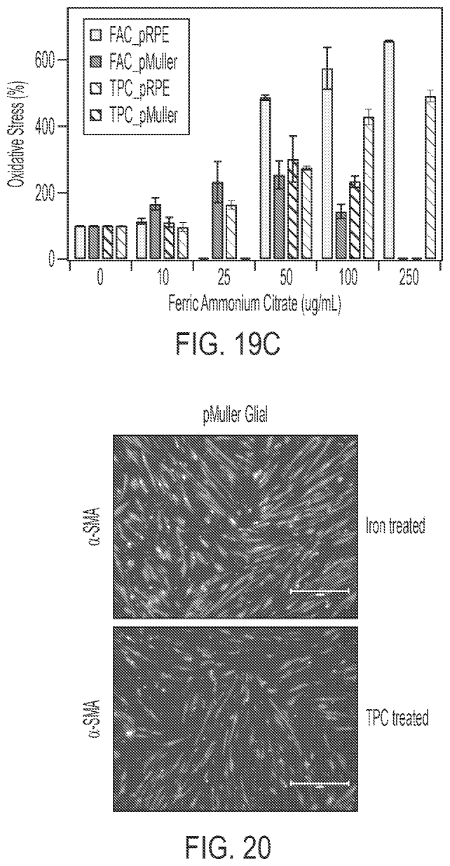

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes-from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As used herein, “administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intra asternal, intrathecal, intraperitoneal, intrahepatic, intralesional, and intracranial injections or infusion techniques), and the like. “Systemic administration” refers to the introducing or delivery to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically effective amount. For example, locally administered gents are easily detectable in the local vicinity of the point of administration but are undetectable or detectable at negligible amounts in distal parts of the subject's body. Administration includes self-administration and the administration by another.

Disclosed herein are compositions including particles including a therapeutic protein, wherein the therapeutic protein includes haptoglobin, hemopexin, transferrin, or a combination thereof.

Haptoglobin, hemopexin, and transferrin may be obtained using a tangential flow filtration of blood, for example plasma or fraction thereof. Methods of such purifications are disclosed in U.S. application Ser. No. 17/427,547, published as US 2022/0098234.

In certain implementations the compositions are substantially free of immunogenic proteins, for example antibodies. As used herein, substantially free of immunogenic proteins indicates the composition includes less than 0.5 wt. %, less than 0.1 wt. %, or less than 0.05 wt. % of immunogenic proteins.

In certain implementations, the compositions can include albumin. In other implementations, the compositions are substantially free of albumin. As used herein, substantially free of albumin indicates the composition includes less than 0.5 wt. %, less than 0.1 wt. %, or less than 0.05 wt. % of albumin.

In some implementations the therapeutic protein includes haptoglobin.

In some implementations, the haptoglobin has an average molecular weight of from 50-100 kDa. In some implementations, the haptoglobin is characterized by having residual hemoglobin as characterized by UV-visible spectroscopy of the Soret peak ranging from 402-407 nm.

In some implementations the therapeutic protein consists of haptoglobin, that is, haptoglobin is the only therapeutic protein in the particle.

In some implementations the therapeutic protein includes hemopexin.

In some implementations the therapeutic protein consists of hemopexin, that is, hemopexin is the only therapeutic protein in the particle.

In some implementations the therapeutic protein includes transferrin.

In some implementations the therapeutic protein consists of transferrin, that is, transferrin is the only therapeutic protein in the particle.

In some implementations the therapeutic protein includes haptoglobin and hemopexin.

In some implementations the therapeutic protein consists of haptoglobin and hemopexin, that is, haptoglobin and hemopexin are the only therapeutic proteins in the particle.

In some implementations the therapeutic protein includes haptoglobin and transferrin.

In some implementations the therapeutic protein consists of haptoglobin and transferrin, that is, haptoglobin and transferrin are the only therapeutic proteins in the particle.

In some implementations the therapeutic protein includes hemopexin and transferrin.

In some implementations the therapeutic protein consists of hemopexin and transferrin, that is, hemopexin and transferrin are the only therapeutic proteins in the particle.

In some implementations the therapeutic protein includes haptoglobin, hemopexin, and transferrin.

In some implementations the therapeutic protein consists of haptoglobin, hemopexin, and transferrin, that is, haptoglobin, hemopexin, and transferrin are the only therapeutic proteins in the particle.

In some implementations the composition further includes water, for example in an amount from 0.1-99 wt. %, from 0.1-1 wt. %, from 1-5 wt. % from 1-10 wt. %, from 1-25 wt. %, from 1-50 wt. %, from 1-75 wt. %, from 1-99 wt. %, from 5-10 wt. %, from 10-25 wt. %, from 10-50 wt. %, from 10-75 wt. %, from 10-99 wt. %, from 25-50 wt. %, from 25-75 wt. %, from 25-99 wt. %, from 50-75 wt. %, from 75-99 wt. %, from 70-80 wt. %, from 70-85 wt. %, from 80-90 wt. %, from 80-95 wt. %., from 90-95 wt. % or from 90-99 wt. %.

In some implementations the composition includes haptoglobin in an amount from 1-200,000 μg/mL, from 1-100,000 μg/mL, from 1-50,000 μg/mL, from 1-10,000 μg/mL, from 1-1,000 μg/ml, from 1,000-10,000 μg/mL, from 5,000-10,000 μg/ml, from 1,000-5,000 μg/mL, from 1,000-2,500 μg/ml, from 500-1,000 μg/ml, from 1-500 μg/mL, or from 500-2,500 μg/ml, relative to the total composition.

In some implementations the composition includes hemopexin in an amount from 1-200,000 μg/mL, from 1-100,000 μg/mL, from 1-50,000 μg/mL, from 1-10,000 μg/mL, from 1-1,000 μg/ml, from 1,000-10,000 μg/mL, from 5,000-10,000 μg/ml, from 1,000-5,000 μg/mL, from 1,000-2,500 μg/ml, from 500-1,000 μg/ml, from 1-500 μg/mL, or from 500-2,500 μg/ml, relative to the total composition. In some implementations the composition includes transferrin in an amount from 1-200,000 μg/mL, from 1-100,000 μg/mL, from 1-50,000 μg/mL, from 1-10,000 μg/mL, from 1-1,000 μg/ml, from 1,000-10,000 μg/mL, from 5,000-10,000 μg/ml, from 1,000-5,000 μg/mL, from 1,000-2,500 μg/ml, from 500-1,000 μg/ml, from 1-500 μg/mL, or from 500-2,500 μg/ml, relative to the total composition.

In some implementations the composition includes haptoglobin and hemopexin in a combined amount from 1-200,000 μg/mL, from 1-100,000 μg/mL, from 1-50,000 μg/mL, from 1-10,000 μg/mL, from 1-1,000 μg/ml, from 1,000 -10,000μg/mL, from 5,000-10,000 μg/ml, from 1,000-5,000 μg/mL, from 1,000-2,500 μg/ml, from 500-1,000 μg/ml, from 1-500 μg/mL, or from 500-2,500 μg/ml, relative to the total composition.

In some implementations the composition includes haptoglobin and transferrin in a combined amount from 1-200,000 μg/mL, from 1-100,000 μg/mL, from 1-50,000 μg/mL, from 1-10,000 μg/mL, from 1-1,000 μg/ml, from 1,000-10,000 μg/mL, from 5,000-10,000 μg/ml, from 1,000-5,000 μg/mL, from 1,000-2,500 μg/ml, from 500-1,000 μg/ml, from 1-500 μg/mL, or from 500-2,500 μg/ml, relative to the total composition.

In some implementations the composition includes hemopexin and transferrin in a combined amount from 1-200,000 μg/mL, from 1-100,000 μg/mL, from 1-50,000 μg/mL, from 1-10,000 μg/mL, from 1-1,000 μg/ml, from 1,000-10,000 μg/mL, from 5,000-10,000 μg/ml, from 1,000-5,000 μg/mL, from 1,000-2,500 μg/ml, from 500-1,000 μg/ml, from 1-500 μg/mL, or from 500-2,500 μg/ml, relative to the total composition.

In some implementations the composition includes haptoglobin, hemopexin, and transferrin in a combined amount from 1-200,000 μg/mL, from 1-100,000 μg/mL, from 1-50,000 μg/mL, from 1-10,000 μg/mL, from 1-1,000 μg/ml, from 1,000-10,000 μg/mL, from 5,000-10,000 μg/ml, from 1,000-5,000 μg/mL, from 1,000-2,500 μg/ml, from 500-1,000 μg/ml, from 1-500 μg/mL, or from 500-2,500 μg/ml, relative to the total composition.

In some implementations the composition includes haptoglobin in an amount from 2-98 wt. %, from 5 -95 wt. %, from 15-75 wt. %, from 15-50 wt. %, from 20-40 wt. %, or from 30-35 wt. %, relative to the total amount of therapeutic protein.

In some implementations the composition includes hemopexin in an amount from 2-98 wt. %, from 5 -95 wt. %, from 15-75 wt. %, from 15-50 wt. %, from 20-40 wt. %, or from 30-35 wt. %, relative to the total amount of therapeutic protein.

In some implementations the composition includes transferrin in an amount from 2-98 wt. %, from 5 -95 wt. %, from 15-75 wt. %, from 15-50 wt. %, from 20-40 wt. %, or from 30-35 wt. %, relative to the total amount of therapeutic protein.

In some implementations the composition includes haptoglobin, hemopexin, and transferrin in about a 1:1:1 mass ratio, about a 2:1:1 mass ratio, about a 1:2:1 mass ratio, about a 1:1:2 mass ratio, about a 2:2:1 mass ratio, about a 1:2:2 mass ratio, about a 2:1:2 mass ratio, about a 3:1:1 mass ratio, about a 1:3:1 mass ratio, about a 1:1:3 mass ratio, about a 3:3:1 mass ratio, about a 1:3:3 mass ratio, or about a 3:1:3 mass ratio.

In some implementations the composition of the haptoglobin is human haptoglobin, in some instances recombinant human haptoglobin.

In some implementations the composition of the hemopexin is human hemopexin, in some instances recombinant human hemopexin.

In some implementations the composition of the transferrin is human transferrin, in some instances recombinant human transferrin.

In some implementations the composition includes particles such as polydopamine particles, polyethylene glycol particles, chitosan particles, poly-(lactic-co-glycolic acid) particle, polylactic acid particles, gold particles, silver particles, MOF particles, cerium oxide particles, silica particles, liposomal particles, poly(meth)acrylate particles, lipid particles, poly(caprolactone) particles, poly(ethyleneimine) particles, protein particles, hyaluronan particles, structured lipids, or a combination thereof.

In some implementations the composition includes particles such as haptoglobin particles, hemopexin particles, transferrin particles, gelatin particles, collagen particles, or a combination thereof.

In certain implementations, the composition includes polydopamine particles. Polydopamine is formed by the oxidation of dopamine. It is biomimetic of the proteins on the extremity of mussel byssus which are extremely right in L-DOPA and L-Lysine residues. These amino acid residues, containing catechol and amino functional groups, allow for strong adhesion of the mussel to all kinds of substrates in the wet and slightly basic environment of sea water. Because of this, polydopamine has traditionally found extensive use in adhesive coatings. Methods for forming polydopamine nanoparticles are known in the art. Typically, polydopamine nanoparticles are formed alkaline aqueous solutions (for example, in the presence of Tris buffer at pH=8.5) in the presence of an oxidant. Oxygen dissolved in the aqueous solution is typically used as the oxidant, but other oxidants may be used, for example ammonium peroxodisulfate or sodium periodate. In some embodiments, the polydopamine nanoparticles as used in the present disclosure are essentially spherical, spheroid, ellipsoid, or combinations thereof.

In some implementations the composition includes the therapeutic protein in an amount from 0.1-100 wt. %, from 0.1-95 wt. %, from 0.1-85 wt. %, from 0.1-75 wt. %, from 1-75 wt. %, from 10-75 wt. %, from 10-50 wt. %, from 10-25 wt. %, from 25-50 wt. %, from 25-75 wt. %, from 50-75 wt. %, from 90-100 wt. %, from 95-100 wt. %, from 90-95 wt. %, from 75-100 wt. %, from 75-95 wt. %, from 30-45 wt. %, from 30-60 wt. %, from 40-75 wt. %, from 1-10 wt. %, from 1-5 wt. %, from 5-10 wt. %, from 5 -15 wt. %, or from 15-30 wt. %, relative to the total weight of the particles.

In some implementations the composition includes particles consisting of haptoglobin, that is the haptoglobin protein forms the particle body. In some implementations other therapeutic proteins may be entrapped or associated within the haptoglobin particle.

In some implementations the composition includes particles consisting of hemopexin, that is the hemopexin protein forms the particle body. In some implementations other therapeutic proteins may be entrapped or associated within the hemopexin particle.

In some implementations the composition includes particles consisting of transferrin, that is the transferrin protein forms the particle body. In some implementations other therapeutic proteins may be entrapped or associated within the transferrin particle.

In some implementations the composition includes particles consisting of haptoglobin and hemopexin, that is the haptoglobin and hemopexin proteins form the particle body. In some implementations other therapeutic proteins may be entrapped or associated within the haptoglobin and hemopexin particle.

In some implementations the composition includes particles consisting of haptoglobin and transferrin, that is the haptoglobin and transferrin proteins form the particle body. In some implementations other therapeutic proteins may be entrapped or associated within the haptoglobin and transferrin particle.

In some implementations the composition includes particles consisting of hemopexin and transferrin, that is the hemopexin and transferrin proteins form the particle body. In some implementations other therapeutic proteins may be entrapped or associated within the hemopexin and transferrin particle.

In some implementations the composition includes particles consisting of haptoglobin, hemopexin, and transferrin.

In some implementations the composition includes the particles in an amount from 1-5,000 μg/mL, from 1-2,500 μg/mL, from 1-1,000 μg/mL, from 1-500 μg/mL, from 1-250 μg/mL, from 1-100 μg/mL, from 1-50 μg/mL, from 1-25 μg/mL, from 1-10 μg/ml, from 5-25 μg/mL, from 10-50 μg/mL, from 25-50 μg/ml, from 25-75 μg/mL, from 25-100 μg/mL, from 50-100 μg/ml, from 75-100 μg/mL, from 75-125 μg/mL, from 50-150 μg/mL, from 100-250 μg/ml, from 250-500 μg/ml, from 500-1,000 μg/mL, from 1,000-2,500 μg/mL, from 1,000-5,000 μg/mL, or from 2,500-5,000 μg/mL.

In some implementations the particles have an average particle size from 1-500,000 nm, from 1-250,000 nm, 1-100,000 nm, from 1-50,000 nm, from 1-25,000 nm, from 1-10,000 nm, from 1-5,000 nm, from 1-1,000 nm, from 1-500 nm, from 1-250 nm, from 1-100 nm, from 1-50 nm, from 50-500 nm, from 50 -250 nm, from 100-500 nm, from 100-250 nm, from 250-500, from 500-750 nm, from 750-1,000 nm, from 500-5,000 nm, from 500-10,000 nm, from 1,000-5,000 nm, from 2,500-5,000 nm, from 1,000-10,000 nm, from 2,500-10,000 nm, from 5,000-10,000 nm, from 10,000-25,000 nm, from 10,000-50,000, from 25,000-50,000, from 25,000-75,000 nm, from 50,000-100,000, or from 50,000 -150,000 nm.

In some implementations the particles are polydopamine particles having an average particle size ranging from about 10 nm to about 1000 nm, for example from about 100 nm to about 1000 nm, from about 200 nm to about 1000 nm, from about 300 nm to about 1000 nm, from about 400 nm to about 1000 nm, from about 500 nm to about 1000 nm, from about 600 nm to about 1000 nm, from about 700 nm to about 1000 nm, from about 800 nm to about 1000 nm, from about 900 nm to about 1000 nm, from about 10 nm to about 900 nm, from about 100 nm to about 900 nm, from about 200 nm to about 900 nm, from about 300 nm to about 900 nm, from about 400 nm to about 900 nm, from about 500 nm to about 900 nm, from about 600 nm to about 900 nm, from about 700 nm to about 900 nm, from about 800 nm to about 900 nm, from about 10 nm to about 800 nm, from about 100 nm to about 800 nm, from about 200 nm to about 800 nm, from about 300 nm to about 800 nm, from about 400 nm to about 800 nm, from about 500 nm to about 800 nm, from about 600 nm to about 800 nm, from about 700 nm to about 800 nm, from about 10 nm to about 700 nm, from about 100 nm to about 700 nm, from about 200 nm to about 700 nm, from about 300 nm to about 700 nm, from about 400 nm to about 700 nm, from about 500 nm to about 700 nm, from about 600 nm to about 700 nm, from about 10 nm to about 600 nm, from about 100 nm to about 600 nm, from about 200 nm to about 600 nm, from about 300 nm to about 600 nm, from about 400 nm to about 600 nm, from about 500 nm to about 600 nm, from about 10 nm to about 500 nm, from about 100 nm to about 500 nm, from about 200 nm to about 500 nm, from about 300 nm to about 500 nm, from about 400 nm to about 500 nm, from about 10 nm to about 400 nm, from about 100 nm to about 400 nm, from about 200 nm to about 400 nm, from about 300 nm to about 400 nm, from about 10 nm to about 300 nm, from about 100 nm to about 300 nm, from about 200 nm to about 300 nm, from about 10 nm to about 200 nm, from about 100 nm to about 200 nm, or from about 10 nm to about 100 nm.

In certain implementations the polydopamine nanoparticles have an average particle size of about 10 nm, about 25 nm, about 50 nm, about 75 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm.

In some embodiments, the polydopamine particles have a loading efficiency of the therapeutic protein(s) of greater than 0.1%, for example greater than 0.5%, greater than 1%, greater than 5%, greater than 10%, greater than 15%, greater than 20%, greater than 25%, greater than 30%, greater than 35%, greater than 40%, greater than 45%, or greater than 50%. In some embodiments, the therapeutic protein(s) may be loaded in the polydopamine nanoparticles in an amount of about 10μg per mg, about 20 μg per mg, about 30 μg per mg, about 40 μg per mg, about 50 μg per mg, about 60 μg per mg, about 70 μg per mg, about 80 μg per mg, about 90 μg per mg, about 100 μg per mg, about 110 μg per mg, about 120 μg per mg, about 130 μg per mg, about 140 μg per mg, about 150 μg per mg, about 160 μg per mg, about 170 μg per mg, about 180 μg per mg, about 190 μg per mg, about 200 μg per mg, about 225 μg per mg, about 250 μg per mg, about 275 μg per mg, about 300 μg per mg, about 325 μg per mg, about 350 μg per mg, about 375 μg per mg, about 400 μg per mg, about 425 μg per mg, about 450 μg per mg, about 475 μg per mg, about 500 μg per mg, or more.

Also disclosed herein are methods of treating or reducing ferroptosis in a subject in need thereof, comprising administering to the subject a therapeutic protein as disclosed herein. In some implementations the therapeutic proteins are loaded onto particles as disclosed herein. Ferroptosis refers to iron-mediated cell death and relates to conditions associated with abnormal iron levels in a given tissue system. The therapeutic protein(s) are effective to reduce inflammation and can be used to treat conditions associated with inflammation.

In some implementations, the therapeutic protein(s) can be used to treat cancer, especially pancreatic cancer, hepatocellular carcinoma, gastric cancer, colorectal cancer, breast cancer, lung cancer, clear cell renal cell carcinoma, adrenocortical carcinoma, ovarian cancer, melanoma, and head and neck cancers). In some implementations the therapeutic protein(s) can be used to treat neurodegenerative disorders, especially Parkinson's disease, Huntington's disease, amyotrophic lateral sclerosis, Friedreich's ataxia, periventricular leukomalacia.

In some implementations the therapeutic protein(s) can be used to treat stroke, for example hemorrhagic stroke. In some implementations the therapeutic protein(s) can be used to treat ischemia/reperfusion injury, for instance following myocardial injury.

In certain implementations the therapeutic protein(s) can be used to treat an autoimmune disease.

In some implementations the therapeutic protein(s) can be used to treat traumatic brain injury.

In some implementations, the therapeutic protein(s) can be used to promote healing in injured tissues. In certain implementations the therapeutic protein(s) can be used to treat injured ocular tissue. In some implementations the therapeutic protein(s) can be used to treat injured cerebrovascular tissue.

Also disclosed herein a method of treating a disorder of the eye by administering to a subject in need thereof a therapeutic protein as disclosed herein. In certain implementations the therapeutic protein(s) can be used to treat an eye disorder such as optic neuropathy, keratopathy, cataract, glaucoma, retinal ischemia-reperfusion injury, age-related macular degeneration, retinitis pigmentosa, diabetic retinopathy, retinoblastoma, thyroid eye disease, degenerative retinal disease, or a combination thereof.

In certain implementations the therapeutic protein(s) can be used to treat retinal detachment, traumatic optic neuropathy, traumatic brain injury, or a combination thereof.

In certain implementations the therapeutic protein(s) can be used to prevent or reduce fibrosis, for example proliferative vitreoretinopathy.

When used in the therapeutic methods disclosed herein, the therapeutic protein(s) may be administered in compositions including particles as disclosed herein. In other implementations, the therapeutic protein(s) may be used in non-particulate compositions. As used herein, a non-particulate composition is a composition wherein the therapeutic protein(s) are not part of a particle system (e.g., loaded within particles or forming particles having a diameter greater than 1 nm) themselves.

In certain implementations, the composition can be injected into the injured tissue, injected intravenously, injected adjacent to the injured tissue (i.e., into a region such that the therapeutic proteins can reach the desired tissue through diffusion or active transport), or a combination thereof.

In some implementations, the composition is injected intraocularly, for example intravitreally (i.e., directly into the vitreous cavity), suprachoroidally (i.e., into the suprachoroidal space between the sclera and the choroid), or retrobulbarly (i.e., into the retrobulbar space, located behind the globe of the eye).

The present disclosure further provides methods of treating an ophthalmological disease or disorder by administering a therapeutically effective amount of the therapeutic protein(s) described herein. In some embodiments, the disclosed methods pertain to treatment of an ophthalmological disorder comprising injecting a therapeutically effective amount of the disclosed therapeutic protein(s) into the eye of a subject. The subject can be a patient; and the patient can have been diagnosed with an ophthalmological disorder. In some aspects, the method can further comprise diagnosing a subject with an ophthalmological disorder.

The ophthalmological disorder can be acute macular neuroretinopathy; Behcet's disease; neovascularization, including choroidal neovascularization; diabetic uveitis; histoplasmosis; infections, such as fungal or viral-caused infections; macular degeneration, such as acute macular degeneration (AMD), including wet AMD, non-exudative AMD and exudative AMD; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion; a posterior ocular condition caused by or influenced by an ocular laser treatment; posterior ocular conditions caused by or influenced by a photodynamic 14 therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, a cancer, and glaucoma. In certain instances, the ophthalmological disorder is wet age-related macular degeneration (wet AMD), a cancer, neovascularization, macular edema, or edema. In a further particular aspect, the ophthalmological disorder is wet age-related macular degeneration (wet AMD).

In various aspects, the injection for treatment of an ophthalmological disorder can be injection to the vitreous chamber of the eye. In some cases, the injection is an intravitreal injection, a subconjunctival injection, a subtenon injection, a retrobulbar injection, or a suprachoroidal injection.

“Ocular region” or “ocular site” means any area of the ocular globe (eyeball), including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include, but are not limited to, the anterior chamber, the posterior chamber, the vitreous cavity, the choroid, the suprachoroidal space, the conjunctiva, the subconjunctival space, the episcleral space, the intracorneal space, the subretinal space, sub-Tenon's space, the epicorneal space, the sclera, the pars plana, surgically-induced avascular regions, the macula, and the retina.

“Ophthalmological disorder” can mean a disease, ailment or condition which affects or involves the eye or one of the parts or regions of the eye. Broadly speaking, the eye includes the eyeball, including the cornea, and other tissues and fluids which constitute the eyeball, the periocular muscles (such as the oblique and rectus muscles) and the portion of the optic nerve which is within or adjacent to the eyeball.

“Glaucoma” means primary, secondary and/or congenital glaucoma. Primary glaucoma can include open angle and closed angle glaucoma. Secondary glaucoma can occur as a complication of a variety of other conditions, such as injury, inflammation, pigment dispersion, vascular disease and diabetes. The increased pressure of glaucoma causes blindness because it damages the optic nerve where it enters the eye. Thus, in one nonlimiting embodiment, by lowering reactive oxygen species, STC-1, or MSCs which express increased amounts of STC-1, may be employed in the treatment of glaucoma and prevent or delay the onset of blindness.

Inflammation-mediated” in relation to an ocular condition means any condition of the eye which can benefit from treatment with an anti-inflammatory agent, and is meant to include, but is not limited to, uveitis, macular edema, acute macular degeneration, retinal detachment, ocular tumors, fungal or viral infections, multifocal choroiditis, diabetic retinopathy, uveitis, proliferative vitreoretinopathy (PVR), sympathetic ophthalmia, Vogt-Koyanagi-Harada (VKH) syndrome, histoplasmosis, and uveal diffusion.

“Injury” or “damage” in relation to an ocular condition are interchangeable and refer to the cellular and morphological manifestations and symptoms resulting from an inflammatory-mediated condition, such as, for example, inflammation, as well as tissue injuries caused by means other than inflammation, such as chemical injury, including chemical burns, as well as injuries caused by infections, including but not limited to, bacterial, viral, or fungal infections.

“Intraocular” means within or under an ocular tissue. An intraocular administration of an ocular therapeutic composition includes administration of the ocular therapeutic composition to a subtenon, subconjunctival, suprachoroidal, subretinal, intravitreal, anterior chamber, and the like location. An intraocular administration of an ocular therapeutic composition excludes administration of the drug delivery system to a topical, systemic, intramuscular, subcutaneous, intraperitoneal, and the like location.

“Macular degeneration” refers to any of a number of disorders and conditions in which the macula degenerates or loses functional activity. The degeneration or loss of functional activity can arise as a result of, for example, cell death, decreased cell proliferation, loss of normal biological function, or a combination of the foregoing. Macular degeneration can lead to and/or manifest as alterations in the structural integrity of the cells and/or extracellular matrix of the macula, alteration in normal cellular and/or extracellular matrix architecture, and/or the loss of function of macular cells. The cells can be any cell type normally present in or near the macula including RPE cells, photoreceptors, and capillary endothelial cells. Age-related macular degeneration, or ARMD, is the major macular degeneration related condition, but a number of others are known including, but not limited to, Best macular dystrophy, Stargardt macular dystrophy, Sorsby fundus dystrophy, Mallatia Leventinese, Doyne honeycomb retinal dystrophy, and RPE pattern dystrophies. Age-related macular degeneration (AMD) is described as either “dry” or “wet.” The wet, exudative, neovascular form of AMD affects about 10-20% of those with AMD and is characterized by abnormal blood vessels growing under or through the retinal pigment epithelium (RPE), resulting in hemorrhage, exudation, scarring, or serous retinal detachment. Eighty to ninety percent of AMD patients have the dry form characterized by atrophy of the retinal pigment epithelium and loss of macular photoreceptors. Drusen may or may not be present in the macula. There may also be geographic atrophy of retinal pigment epithelium in the macula accounting for vision loss. At present there is no cure for any form of AMD, although some success in attenuation of wet AMD has been obtained with photodynamic and especially anti-VEGF therapy.

“Drusen” is debris-like material that accumulates with age below the RPE. Drusen is observed using a funduscopic eye examination. Normal eyes may have maculas free of drusen, yet drusen may be abundant in the retinal periphery. The presence of soft drusen in the macula, in the absence of any loss of macular vision, is considered an early stage of AMD. Drusen contains a variety of lipids, polysaccharides, and glycosaminoglycans along with several proteins, modified proteins or protein adducts. There is no generally accepted therapeutic method that addresses drusen formation and thereby manages the progressive nature of AMD.

“Ocular neovascularization” (ONV) is used herein to refer to choroidal neovascularization or retinal neovascularization, or both.

“Retinal neovascularization” (RNV) refers to the abnormal development, proliferation, and/or growth of retinal blood vessels, e.g., on the retinal surface.

“Subretinal neovascularization” (SRNVM) refers to the abnormal development, proliferation, and/or growth of blood vessels beneath the surface of the retina.

“Cornea” refers to the transparent structure forming the anterior part of the fibrous tunic of the eye. It consists of five layers, specifically: 1) anterior corneal epithelium, continuous with the conjunctiva; 2) anterior limiting layer (Bowman's layer); 3) substantia propria, or stromal layer; 4) posterior limiting layer (Descemet's membrane); and 5) endothelium of the anterior chamber or keratoderma.

“Retina” refers to the innermost layer of the ocular globe surrounding the vitreous body and continuous posteriorly with the optic nerve. The retina is composed of layers including the: 1) internal limiting membrane; 2) nerve fiber layer; 3) layer of ganglion cells; 4) inner plexiform layer; 5) inner nuclear layer; 6) outer plexiform layer; 7) outer nuclear layer; 8) external limiting membrane; and 9) a layer of rods and cones.

“Retinal degeneration” refers to any hereditary or acquired degeneration of the retina and/or retinal pigment epithelium. Non-limiting examples include retinitis pigmentosa, Best's Disease, RPE pattern dystrophies, and age-related macular degeneration.

In various aspects, a method of treating an ophthalmological disorder may comprise treatment of various ocular diseases or conditions of the retina, including the following:
maculopathies/retinal degeneration: macular degeneration, including age-related macular degeneration (ARMD), such as non-exudative age-related macular degeneration and exudative age-related macular degeneration; choroidal neovascularization; retinopathy, including diabetic retinopathy, acute and chronic macular neuroretinopathy, central serous chorioretinopathy; and
macular edema, including cystoid macular edema, and diabetic macular edema.
Uveitis/retinitis/choroiditis: acute multifocal placoid pigment epitheliopathy, Behcet's disease, birdshot retinochoroidopathy, infectious (syphilis, Lyme Disease, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coats disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease, Traumatic/surgical diseases: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy.
Infectious disorders: ocular histoplasmosis, ocular toxocariasis, ocular histoplasmosis syndrome (OHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Best's disease, pattern dystrophy of the retinal pigment epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigment epithelium, retinoblastoma, vasoproliferative tumors of the ocular fundus, retinal astrocytoma, and intraocular lymphoid tumors. Miscellaneous: punctate inner choroidopathy, acute posterior multifocal placoid pigment epitheliopathy, myopic retinal degeneration, acute retinal pigment epithelitis and the like.

An anterior ocular condition is a disease, ailment or condition which affects or which involves an anterior (i.e., front of the eye) ocular region or site, such as a periocular muscle, an eyelid or an eyeball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; posterior capsule opacification (PCO); conjunctival diseases; conjunctivitis, including, but not limited to, atopic keratoconjunctivitis; corneal injuries, including, but not limited to, injury to the corneal stromal areas; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

Other diseases or disorders of the eye which may be treated in accordance with the present invention include, but are not limited to, ocular cicatricial pemphigoid (OCP), Stevens Johnson syndrome and cataracts.

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e., the optic disc), and blood vessels and nerves which vascularize or innervate a posterior ocular region or site. Thus, a posterior ocular condition can include a disease, ailment or condition, such as for example, acute macular neuroretinopathy; Behcet's disease; choroidal neovascularization; diabetic retinopathy; uveitis; ocular histoplasmosis; infections, such as fungal or viral-caused infections; macular degeneration, such as acute macular degeneration, non-exudative age-related macular degeneration and exudative age-related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial or venous occlusive disease, retinal detachment, uveitic retinal disease; sympathetic ophthalmia; Vogt-Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal ganglion cells or retinal nerve fibers (i.e., neuroprotection).

In some embodiments, the ophthalmic disorder is ocular inflammation resulting from, e.g., iritis, conjunctivitis, seasonal allergic conjunctivitis, acute and chronic endophthalmitis, anterior uveitis, uveitis associated with systemic diseases, posterior segment uveitis, chorioretinitis, pars planitis, masquerade syndromes including ocular lymphoma, pemphigoid, scleritis, keratitis, severe ocular allergy, corneal abrasion and blood-aqueous barrier disruption. In yet another embodiment, the ophthalmic disorder is post-operative ocular inflammation resulting from, for example, photorefractive keratectomy, cataract removal surgery, intraocular lens implantation, vitrectomy, corneal transplantation, forms of lamellar keratectomy (DSEK, etc.), and radial keratotomy.

In various aspects, the injection for treatment of an ophthalmological disorder can be injection to the vitreous chamber of the eye. In some cases, the injection is an intravitreal injection, a subconjunctival injection, a subtenon injection, a retrobulbar injection, or a suprachoroidal injection.

In certain implementations the composition may be prepared using a physiological saline solution as a vehicle. The pH of the composition may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

Any diluent used in the preparation of the composition may preferably be selected so as not to unduly affect the biological activity of the composition. Example of such diluents which are especially for injectable ophthalmic compositions are water, various saline, organic, or inorganic salt solutions, Ringer's solution, dextrose solution, and Hank's solution.

In addition, the compositions may include additives such other buffers, diluents, carriers, adjuvants, or excipients. Any pharmaceutically acceptable buffer suitable for application to the eye may be used, e.g., tris or phosphate buffers. Other agent may be employed in the formulation for a variety of purposes. For example, buffering agents, preservatives, co-solvents, surfactants, oils, humectants, emollients, chelating agents, stabilizers or antioxidants may be employed. Water soluble preservatives which may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. A surfactant may be Tween 80.

Other vehicles that may be used include, but are not limited to, polyvinyl alcohol, povidone, hydroxypropyl methyl cellulose, poloxamers, carboxymethyl cellulose, hydroxyethyl cellulose, or purified water. Tonicity adjustors may be included, for example, sodium chloride, potassium chloride, mannitol, or glycerin. Antioxidants include, but are not limited to, sodium metabisulfite, sodium thiosulfate, acetylcysteine, butylated hydroxyanisole, or butylated hydroxytoluene. The indications, effective doses, formulations, contraindications, etc. of the components in the composition are available and are known to one skilled in the art.

These agents may be present in individual amounts from about 0.001% to about 5% by weight and preferably about 0.01% to about 2% by weight in the formulation. Suitable water soluble buffering agents that may be employed are sodium carbonate, sodium borate, sodium phosphate, sodium acetate, or sodium bicarbonate, as approved by the U.S. FDA for the desired route of administration. These agents may be present in amounts sufficient to maintain a pH of the system between about 2 to about 9 and preferably about 4 to about 8. As such, the buffering agent may be as much as about 5% (w/w) of the total ocular therapeutic composition. Electrolytes such as, but limited to, sodium chloride and potassium chloride may be also included in the formulation.

In some implementations, the composition further comprises a hydrogel. In some embodiments, the hydrogel comprises a polymer composition, for example a homopolymer, a copolymer, or combinations thereof. In some embodiments, the hydrogel comprises one or more hydrophilic polymers, i.e. a polymer having at least 0.1 wt. % solubility in water, for example having at least 0.5 wt. % solubility. In some embodiments, the hydrophilic polymer has a solubility of at least 1 mg/mL.

In some embodiments, the polymer may comprise one or more vinyl alcohol residues. In some embodiments, the polymer may comprise one or more acrylamide residues. In some embodiments, the polymer may comprise one or more residues selected from a polyethylene glycol derivative or a functionalized polyethylene glycol. In some embodiments, the polymer composition may comprise one or more acrylate residues or one or more methacrylate residues. In some embodiments, the polymer composition may comprise one or more residues selected from acrylamide, N-ornithine acrylamide, N-(2-hydroxypropyl)acrylamide, hydroxyethylacrylate, hydroxyethylmethacrylate, polyethyleneglycol acrylates, polyethylenegylcol methacrylates, N-vinylpyrrolidinone, N-phenylacrylamide, dimethylaminopropyl methacrylamide, acrylic acid, benzylmethacrylamide, methylthioethylacrulamide, or combinations thereof.

Representative examples of hydrogels which can be used include, but are not limited to, hyaluronic acid, collagen, gellan, silk, fibrin, alginate, chitosan, polyacrylamides and methacrylate derivatives thereof, polyacrylic acid and methacrylate derivatives thereof, polyvinyl alcohol, polyethylene glycol and derivatives thereof, polypropylene glycol and derivatives thereof, or combinations thereof.

In some embodiments, the hydrogel comprises a hyaluronate derivative, for example poly(N-isopropylacrylamide) grafted sodium hyaluronate.

In some implementations, the compositions can include one or more additional therapeutic agents beyond the therapeutic proteins disclosed herein. As used herein, a “therapeutic agent” refers to one or more therapeutic agents, active ingredients, or substances that can be used to treat a medical condition of the eye or a cancer. The therapeutic agents are typically ophthalmically acceptable and are provided in a form that does not cause adverse reactions when the compositions disclosed herein are placed in an eye. As discussed herein, the therapeutic agents can be released from the disclosed compositions in a biologically active form. For example, the therapeutic agents may retain their three-dimensional structure when released from the composition into an eye.

It is further understood, that as used herein, the terms “therapeutic agent” includes any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians'Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double-and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

In some embodiments, the therapeutic agent may comprise an agent useful in the treatment of an ophthalmological disorder or an eye disease such as: beta-blockers including timolol, betaxolol, levobetaxolol, and carteolol; miotics including pilocarpine; carbonic anhydrase inhibitors; serotonergics; muscarinics; dopaminergic agonists; adrenergic agonists including apraclonidine and brimonidine; anti-angiogenesis agents; anti-infective agents including quinolones such as ciprofloxacin and aminoglycosides such as tobramycin and gentamicin; nonsteroidal and steroidal anti-inflammatory agents, such as suprofen, diclofenac, ketorolac, rimexolone and tetrahydrocortisol; growth factors, such as EGF; immunosuppressant agents; and anti-allergic agents including olopatadine; prostaglandins such as latanoprost; 15-keto latanoprost; travoprost; and unoprostone isopropyl.

In some embodiments, the therapeutic agent is selected from the group consisting of an anti-inflammatory agent, a calcineurin inhibitor, an antibiotic, a nicotinic acetylcholine receptor agonist, and an anti-lymphangiogenic agent. In some embodiments, the anti-inflammatory agent may be cyclosporine. In some embodiments, the calcineurin inhibitor may be voclosporin. In some embodiments, the antibiotic may be selected from the group consisting of amikacin, gentamycin, kanamycin, neomycin, netilmicin, streptomycin, tobramycin, teicoplanin, vancomycin, azithromycin, clarithromycin, dirithromycin, erythromycin, roxithromycin, troleandomycin, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, sulfacetamide, sulfamethizole, sulfasalazine, sulfisoxazole, trimethoprim, cotrimoxazole, demeclocycline, doxycycline, minocycline, oxytetracycline, and tetracycline. In some embodiments, the nicotinic acetylcholine receptor agonist may be any of pilocarpine, atropine, nicotine, epibatidine, lobeline, or imidacloprid. In some embodiments, the anti-lymphangiogenic agent may be a vascular endothelial growth factor C (VEGF-C) antibody, a VEGF-D antibody or a VEGF-3 antibody.

In some aspects, the therapeutic agent may be selected from: a beta-blocker, including levobunolol (BETAGAN), timolol (BETIMOL, TIMOPTIC), betaxolol (BETOPTIC) and metipranolol (OPTIPRANOLOL); alpha-agonists, such as apraclonidine (IOPIDINE) and brimonidine (ALPHAGAN); carbonic anhydrase inhibitors, such as acetazolamide, methazolamide, dorzolamide (TRUSOPT) and brinzolamide (AZOPT); prostaglandins or prostaglandin analogs such as latanoprost (XALATAN), bimatoprost (LUMIGAN) and travoprost (TRAVATAN); miotic or cholinergic agents, such as pilocarpine (ISOPTO CARPINE, PILOPINE) and carbachol (ISOPTO CARBACHOL); epinephrine compounds, such as dipivefrin (PROPINE); forskolin; or neuroprotective compounds, such as brimonidine and memantine; a steroid derivative, such as 2-methoxyestradiol or analogs or derivatives thereof; or an antibiotic.

The term “anti-RAS agent” or “anti-Renin Angiotensin System agent” refers to refers to an agent that reduces, or inhibits, either partially or fully, the activity or production of a molecule of the renin angiotensin system (RAS). Non-limiting examples of “anti-RAS” or “anti-Renin Angiotensin System” molecules are one or more of an angiotensin-converting enzyme (ACE) inhibitor, an angiotensin-receptor blocker, and a renin inhibitor.

In some embodiments, the therapeutic agent may comprise a renin angiotensin system (RAS) inhibitor. In some embodiments, the renin angiotensin system (RAS) inhibitor is one or more of an angiotensin-converting enzyme (ACE) inhibitor, an angiotensin-receptor blocker, and a renin inhibitor.

Non limiting examples of angiotensin-converting enzyme (ACE) inhibitors which are useful in the present invention include, but are not limited to: alacepril, alatriopril, altiopril calcium, ancovenin, benazepril, benazepril hydrochloride, benazeprilat, benzazepril, benzoylcaptopril, captopril, captoprilcysteine, captoprilglutathione, ceranapril, ceranopril, ceronapril, cilazapril, cilazaprilat, converstatin, delapril, delaprildiacid, enalapril, enalaprilat, enalkiren, enapril, epicaptopril, foroxymithine, fosfenopril, fosenopril, fosenopril sodium, fosinopril, fosinopril sodium, fosinoprilat, fosinoprilic acid, glycopril, hemorphin-4, idapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spirapril hydrochloride, temocapril, temocapril hydrochloride, teprotide, trandolapril, trandolaprilat, utibapril, zabicipril, zabiciprilat, zofenopril, zofenoprilat, pharmaceutically acceptable salts thereof, and mixtures thereof.

Non limiting examples of angiotensin-receptor blockers which are useful in the present invention include, but are not limited to: irbesartan (U.S. Pat. No. 5,270,317, hereby incorporated by reference in its entirety), candesartan (U.S. Pat. Nos. 5,196,444 and 5,705,517 hereby incorporated by reference in their entirety), valsartan (U.S. Pat. No. 5,399,578, hereby incorporated by reference in its entirety), and losartan (U.S. Pat. No. 5,138,069, hereby incorporated by reference in its entirety).

Non limiting examples of renin inhibitors which may be used as therapeutic agents include, but are not limited to: aliskiren, ditekiren, enalkiren, remikiren, terlakiren, ciprokiren and zankiren, pharmaceutically acceptable salts thereof, and mixtures thereof.

The term “steroid” refers to compounds belonging to or related to the following illustrative families of compounds: corticosteroids, mineralicosteroids, and sex steroids (including, for example, potentially androgenic or estrogenic or anti-androgenic and anti-estrogenic molecules). Included among these are, for example, prednisone, prednisolone, methylprednisolone, triamcinolone, fluocinolone, aldosterone, spironolactone, danazol (otherwise known as OPTINA), and others. In some embodiments, the therapeutic agent may comprise a steroid.

The terms “peroxisome proliferator-activated receptor gamma agent,” or “PPAR-y agent,” or “PPARG agent,” or “PPAR-gamma agent” refers to agents which directly or indirectly act upon the peroxisome proliferator-activated receptor. This agent may also influence PPAR-alpha, “PPARA” activity.

In some embodiments, the therapeutic agent may comprise a modulator of macrophage polarization. Illustrative modulators of macrophage polarization include peroxisome proliferator activated receptor gamma (PPAR-g) modulators, including, for example, agonists, partial agonists, antagonists or combined PPAR-gamma/alpha agonists. In some embodiments, the therapeutic agent may comprise a PPAR gamma modulator, including PPAR gamma modulators that are full agonists or a partial agonists. In some embodiments, the PPAR gamma modulator is a member of the drug class of thiazolidinediones (TZDs, or glitazones). By way of non-limiting example, the PPAR gamma modulator may be one or more of rosiglitazone (AVANDIA), pioglitazone (ACTOS), troglitazone (REZULIN), netoglitazone, rivoglitazone, ciglitazone, rhodanine. In some embodiments, the PPAR gamma modulator is one or more of irbesartan and telmesartan. In some embodiments, the PPAR gamma modulator is a nonsteroidal anti-inflammatory drug (NSAID, such as, for example, ibuprofen) or an indole.

Known inhibitors include the experimental agent GW-9662. Further examples of PPAR gamma modulators are described in WIPO Publication Nos. WO/1999/063983, WO/2001/000579, Nat Rev Immunol. 2011 Oct. 25; 11(11): 750-61, or agents identified using the methods of WO/2002/068386, the contents of which are hereby incorporated by reference in their entireties.

In some embodiments, the PPAR gamma modulator is a “dual,” or “balanced,” or “pan” PPAR modulator. In some embodiments, the PPAR gamma modulator is a glitazar, which bind two or more PPAR isoforms, e.g., muraglitazar (Pargluva) and tesaglitazar (Galida) and aleglitazar.

In some embodiments, the therapeutic agent may comprise semapimod (CNI-1493) as described in Bianchi, et al. (March 1995). Molecular Medicine (Cambridge, Mass.) 1 (3): 254-266, the contents of which is hereby incorporated by reference in its entirety.

In some embodiments, the therapeutic agent may comprise a migration inhibitory factor (MIF) inhibitor. Illustrative MIF inhibitors are described in WIPO Publication Nos. WO 2003/104203, WO 2007/070961, WO 2009/117706 and U.S. Pat. Nos. 7,732,146 and 7,632,505, and 7,294,753 7,294,753 the contents of which are hereby incorporated by reference in their entireties. In some embodiments, the MIF inhibitor is (S, R)-3-(4-hydroxyphenyl)-4,5-dihydro-5-isoxazole acetic acid methyl ester (ISO-1), isoxazoline, p425 (J. Biol. Chem., 287, 30653-30663), epoxyazadiradione, or vitamin E.

In some embodiments, the therapeutic agent may comprise a chemokine receptor 2(CCR2 ) inhibitor as described in, for example, U.S. patent and Patent Publication Nos.: U.S. Pat. Nos. 7,799,824, 8,067,415, US 2007/0197590, US 2006/0069123, US 2006/0058289, and US 2007/0037794, the contents of which are hereby incorporated by reference in their entireties. In some embodiments, the CCR2) inhibitor is Maraviroc, cenicriviroc, CD192, CCX872,CCX140, 2-((Isopropylaminocarbonyl)amino)-N-(2-((cis-2-((4-(methylthio)benzoyl)amino)cyclohexyl)amino)-2-oxoethyl)-5-(trifluoromethyl)-benzamide, vicriviroc, SCH351125, TAK779, Teijin, RS-504393, compound 2, compound 14, or compound 19 (PLOS ONE 7(3): e32864).

In some embodiments, the therapeutic agent may comprise an agent that modulates autophagy, microautophagy, mitophagy or other forms of autophagy. In some embodiments, the therapeutic agent may comprise sirolimus, tacrolimis, rapamycin, everolimus, bafilomycin, chloroquine, hydroxychloroquine, spautin-1, metformin, perifosine, resveratrol, trichostatin, valproic acid, Z-VAD-FMK, or others known to those in the art.

According to certain aspects, the therapeutic agent may comprise a biologic drug, particularly an antibody. According to some aspects, the antibody is selected from the group consisting of cetuximab, anti-CD24 antibody, panitumumab and bevacizumab.

Therapeutic agents as used in the present disclosure may comprise peptides, proteins such as hormones, enzymes, antibodies, monoclonal antibodies, antibody fragments, monoclonal antibody fragments, and the like, nucleic acids such as aptamers, siRNA, DNA, RNA, antisense nucleic acids or the like, antisense nucleic acid analogs or the like, low-molecular weight compounds, or high-molecular-weight compounds, receptor agonists, receptor antagonists, partial receptor agonists, and partial receptor antagonists.

Additional representative therapeutic agents may include, but are not limited to, peptide drugs, protein drugs, desensitizing materials, antigens, factors, growth factors, anti-infective agents such as antibiotics, antimicrobial agents, antiviral, antibacterial, antiparasitic, antifungal substances and combination thereof, antiallergenics, steroids, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, anti-Alzheimer's agents, antihypertensive agents, beta-adrenergic blocking agents, alpha-adrenergic blocking agents, nutritional agents, and the benzophenanthridine alkaloids. The therapeutic agent can further be a substance capable of acting as a stimulant, a sedative, a hypnotic, an analgesic, an anticonvulsant, and the like.

Additional therapeutic agents may comprise CNS-active drugs, neuro-active drugs, inflammatory and anti-inflammatory drugs, renal and cardiovascular drugs, gastrointestinal drugs, antineoplastics, immunomodulators, immunosuppressants, hematopoietic agents, growth factors, anticoagulant, thrombolytic, antiplatelet agents, hormones, hormone-active agents, hormone antagonists, vitamins, ophthalmic agents, anabolic agents, antacids, anti-asthmatic agents, anti-cholesterolemic and anti-lipid agents, anti-convulsants, anti-diarrheals, anti-emetics, anti-manic agents, antimetabolite agents, anti-nauseants, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, laxatives, mineral supplements, mucolytic agents, neuromuscular drugs, peripheral vasodilators, psychotropics, stimulants, thyroid and anti-thyroid agents, tissue growth agents, uterine relaxants, vitamins, antigenic materials, and so on. Other classes of therapeutic agents include those cited in Goodman & Gilman's The Pharmacological Basis of Therapeutics (McGraw Hill) as well as therapeutic agents included in the Merck Index and The Physicians'Desk Reference (Thompson Healthcare).

Other therapeutic agents include androgen inhibitors, polysaccharides, growth factors (e.g., a vascular endothelial growth factor-VEGF), hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Further examples of therapeutic agents include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, antiproliferatives, anti-VEGF agents, cardioactive agents, nonsteroidal anti-inflammatory agents, antiparkinsonian agents, antihypertensive agents, β-adrenergic blocking agents, nutritional agents, and the benzophenanthridine alkaloids. The agent can further be a substance capable of acting as a stimulant, sedative, hypnotic, analgesic, anticonvulsant, and the like.

Further representative therapeutic agents include but are not limited to analgesics such as acetaminophen, acetylsalicylic acid, and the like; anesthetics such as lidocaine, xylocaine, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, paclitaxel, taxol, and the like; anticoagulants such as heparin, warfarin, and the like; anticonvulsants such as phenyloin sodium, diazepam, and the like; antidepressants such as isocarboxazid, amoxapine, and the like; antihistamines such as diphenhydramine HCI, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B12, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; antimalarials, L-dopa, diuretics such as furosemide, spironolactone, and the like; antiulcer drugs such as rantidine HCI, cimetidine HCl, and the like.

The therapeutic agent can also be an immunomodulator, including, for example, cytokines, interleukins, interferon, colony stimulating factor, tumor necrosis factor, and the like; immunosuppressants such as rapamycin, tacrolimus, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcus neoformans, Histoplasma capsulatum, Candida albicans, Candida tropicalis, Nocardia asteroids, Rickettsia ricketsii, Rickettsia typhi, Mycoplasma pneumoniae, Chlamydia psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the therapeutic agent can comprise an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Timidazole, or a combination thereof. In one aspect, the therapeutic agent can be a combination of Rifampicin (Rifampin in U.S.) and Minocycline.

Growth factors useful as therapeutic agents include, but are not limited to, transforming growth factor-α (“TGF-α”), transforming growth factors (“TGF-β”), platelet-derived growth factors (“PDGF”), fibroblast growth factors (“FGF”), including FGF acidic isoforms 1 and 2, FGF basic form 2 and FGF 4, 8, 9 and 10, nerve growth factors (“NGF”) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, beta3, skeletal growth factor, bone matrix derived growth factors, and bone derived growth factors and mixtures thereof.

Cytokines useful as therapeutic agents include, but are not limited to, cardiotrophin, stromal cell derived factor, macrophage derived chemokine (MDC), melanoma growth stimulatory activity (MGSA), macrophage inflammatory proteins 1 alpha (MIP-1alpha), 2, 3 alpha, 3 beta, 4 and 5, IL-1,IL-2, IL-3, IL-4, IL-5, IL-6, IL-7, IL-8, IL-9, IL-10, IL-11, IL-12, IL-13, TNF-α, and TNF-β.

Immunoglobulins useful in the present disclosure include, but are not limited to, IgG, IgA, IgM, IgD, IgE, and mixtures thereof. Some preferred growth factors include VEGF (vascular endothelial growth factor), NGFs (nerve growth factors), PDGF-AA, PDGF-BB, PDGF-AB, FGFb, FGFa, and BGF.

Other molecules useful as therapeutic agents include but are not limited to growth hormones, leptin, leukemia inhibitory factor (LIF), tumor necrosis factor alpha and beta, endostatin, thrombospondin, osteogenic protein-1, bone morphogenetic proteins 2 and 7, osteonectin, somatomedin-like peptide, osteocalcin,, interferon alpha, interferon alpha A, interferon beta, interferon gamma, interferon 1 alpha, and interleukins 2, 3, 4, 5 6, 7, 8, 9, 10, 11, 12, 13, 15, 16, 17 and 18.

In some embodiments, the therapeutic agent comprises a drug used in the treatment of cataract or posterior capsule opacification. In some embodiments, the therapeutic agent comprises a drug used in the treatment of proliferative vitreoretinopathy. In some embodiments, the therapeutic agent comprises a drug used in the treatment of diabetic retinopathy, for example insulin. In some embodiments, the therapeutic agents comprises a drug used in the treatment of uveal melanoma, for example dacarbazine, interferon-alpha, cisplatin, tamoxifen, sunitinib, fotemustine, crizotinib, valproic acid, ipilimumab, nivolumab, or combinations thereof.

### EXAMPLES

The following examples are for the purpose of illustration of the invention only and are not intended to limit the scope of the present invention in any manner whatsoever.

### Hemoglobin (Hb), Heme, and Iron Scavenging Protein Cocktail Purification Via Tangential Flow Filtration (TFF)

Briefly, 500 g of human Cohn Fraction IV was suspended in 5 L of phosphate-buffered saline (PBS) and homogenized in a blender. After stirring overnight at 4° C., approximately 5 L of the solution was centrifuged to remove undissolved lipids. Fumed silica was then added to the supernatant to remove lipids and lipoproteins, left to stir overnight at 4° C., and then recentrifuged with two PBS washes of the fumed silica pellet. The resulting protein solution was then clarified on a 0.2 μm hollow fiber (HF) filter and then bracketed using a series of HF modules with decreasing molecular weight cutoff (MWCO) (750, 500, and 100 kDa). The permeate of the 100 kDa HF filter (Stage 3) was retained on a 50 kDa HF module. The new bracket (Stage 4) was subject to constant volume diafiltration with 5× volume PBS and finally concentrated to approximately 350 ml.

### Hplc-Size Exclusion Chromatography (SEC)

Samples from the purification process were separated via HPLC-SEC using a commercial column (4.6×300 mm Acclaim SEC-1000, Thermo Fisher Scientific, Waltham, MA) attached to an HPLC system (Dionex UltiMate 3000, Thermo Fisher Scientific, Waltham, MA).

### Gel Electrophoresis

The purity and composition of the protein cocktail were analyzed via SDS-PAGE using commercial equipment (Invitrogen Mini Gel Tank, Thermo Fisher Scientific, Waltham, MA). The samples were prepared according to the manufacturer's guidelines.

### Total Protein Assay

Total protein was determined via the Bradford assay.

### Hemoglobin (Hb) Binding Capacity of Hp

The difference in molecular weight (MW) between the Hp-Hb protein complex and pure Hb was used to assess the Hb binding capacity (HbBC) of Hp using HPLC-SEC. Briefly, the samples containing Hp were mixed with excess Hb and then separated via HPLC-SEC. The difference in the area under the curve (AUC) between the pure Hb solution and the mixture of Hb and Hp was used to assess the HbBC of Hp.

### Iron Binding Activity

The iron-binding capacity (FeBC) of Tf contained in the protein scavenger cocktail was determined via reaction with ferric nitrilotriacetate [Fe(NTA)]. Briefly, the Tf sample was reacted with excess Fe(NTA), and the equilibrium change in absorbance was measured. The extinction coefficient of holo-Tf at 465 nm was then used to estimate the concentration of iron bound to Tf (FeBC). The holo-Tf concentration was determined based on the 465 nm absorbance of the sample prior to the addition of Fe(NTA) (contribution of residual metHb in the sample at 465 nm was estimated based on the sample absorbance at 404 nm).

### Heme Binding Activity

The heme-binding capacity (HemeBC) in the purified protein scavenger cocktail was determined via the dicyanohemin (DCNh) incorporation assay. Briefly, the sample was mixed with increasing concentrations of DCNh, and the equilibrium absorbance of the Soret peak maxima was measured. The inflection point in the graph of the equilibrium absorbance versus DCNh concentration was used to determine the saturation point of the heme-binding pockets. To determine the heme-binding activity of Hpx individually, the protein cocktail was mixed with excess heme-bound HSA (hHSA) and the change in absorbance was used to determine the concentration of heme-Hpx.

### Trypsin Digest Mass Spectrometry

Protein identification in the protein cocktail was confirmed using trypsin digest nano-liquid chromatography-nanospray tandem mass spectrometry (LC/MS/MS) on a commercial mass spectrometer (Fusion Orbitrap equipped with EASY-Spray™ Sources, Thermo Scientific, San Jose, CA) operated in positive ion mode.

### ELISA

The concentration of selected protein components in human Cohn Fraction IV and in the protein cocktail was quantified via ELISA kits specific for Hp, Tf, HSA, and Hpx according to the manufacturer's instructions (R&D Systems Catalog #DHAPGO for Hp, and Eagle BioSciences HTF31-K01 for Tf, HUA39-K01 for HSA, and HPX39-K01 for Hpx).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches

1. A composition, comprising particles comprising a therapeutic protein, wherein the therapeutic protein comprises haptoglobin, hemopexin, transferrin, or a combination thereof.

2. The composition according to claim 1, wherein the therapeutic protein comprises haptoglobin, hemopexin, or transferrin.

3-7. (canceled)

8. The composition according to claim 1, wherein the therapeutic protein comprises haptoglobin and hemopexin, haptoglobin and transferrin, or hemopexin and transferrin.

9-13. (canceled)

14. The composition according to claim 1, wherein the therapeutic protein comprises haptoglobin, hemopexin, and transferrin.

15. (canceled)

16. The composition according to claim 1, wherein the composition further comprises water.

17. The composition according to claim 1, comprising haptoglobin, hemopexin, or transferrin in an amount from 1-200,000 μg/mL, from 1-100,000 μg/mL, from 1-50,000 μg/mL, from 1-10,000 μg/mL, from 1-1,000 μg/mL, from 1,000-10,000 μg/mL, from 5,000-10,000 μg/mL, from 1,000-5,000 μg/mL, from 1,000-2,500 μg/mL, from 500-1,000 μg/mL, from 1-500 μg/mL, or from 500-2,500 μg/mL, relative to the total composition.

18-19. (canceled)

20. The composition according to claim 1, comprising haptoglobin and hemopexin, haptoglobin and transferrin, or hemopexin and transferrin in a combined amount from 1-200,000 μg/mL, from 1-100,000 μg/mL, from 1-50,000 μg/mL, from 1-10,000 μg/mL, from 1-1,000 μg/mL, from 1,000-10,000 μg/mL, from 5,000-10,000 μg/mL, from 1,000-5,000 μg/mL, from 1,000-2,500 μg/mL, from 500-1,000 μg/mL, from 1-500 μg/mL, or from 500-2,500 μg/mL, relative to the total composition.

21-22. (canceled)

23. The composition according to claim 1, comprising haptoglobin, hemopexin, and transferrin in a combined amount from 1-200,000 μg/mL, from 1-100,000 μg/mL, from 1-50,000 μg/mL, from 1-10,000 μg/mL, from 1-1,000 μg/mL, from 1,000-10,000 μg/mL, from 5,000-10,000 μg/mL, from 1,000-5,000 μg/mL, from 1,000-2,500 μg/mL, from 500-1,000 μg/mL, from 1-500 μg/mL, or from 500-2,500 μg/mL, relative to the total composition.

24. The composition according to claim 1, comprising haptoglobin, hemopexin, or transferrin in an amount from 2-98 wt. %, from 5 -95 wt. %, from 15-75 wt. %, from 15-50 wt. %, from 20-40 wt. %, or from 30-35 wt. %, relative to the total amount of therapeutic protein.

25-26. (canceled)

27. The composition according to claim 1, comprising haptoglobin, hemopexin, and transferrin in about a 1:1:1 mass ratio, about a 2:1:1 mass ratio, about a 1:2:1 mass ratio, about a 1:1:2 mass ratio, about a 2:2:1 mass ratio, about a 1:2:2 mass ratio, about a 2:1:2 mass ratio, about a 3:1:1 mass ratio, about a 1:3:1 mass ratio, about a 1:1:3 mass ratio, about a 3:3:1 mass ratio, about a 1:3:3 mass ratio, or about a 3:1:3 mass ratio.

28. The composition according to claim 1, wherein the haptoglobin comprises human haptoglobin, or
wherein the hemopexin comprises human hemopexin, or
wherein the transferrin comprises human transferrin.

29-30. (canceled)

31. The composition according to claim 1, wherein the particles comprise polydopamine particles, polyethylene glycol particles, chitosan particles, poly-(lactic-co-glycolic acid) particle, polylactic acid particles, gold particles, silver particles, MOF particles, cerium oxide particles, silica particles, liposomal particles, poly(meth)acrylate particles, lipid particles, poly(caprolactone) particles, poly(ethyleneimine) particles, protein particles, hyaluronan particles, structured lipids, or a combination thereof.

32. The composition according to claim 1, wherein the particles comprise haptoglobin particles, hemopexin particles, transferrin particles, gelatin particles, collagen particles, or a combination thereof.

33. The composition according to claim 1, wherein the particles comprise the therapeutic protein in an amount from 0.1-100 wt. %, from 0.1-95 wt. %, from 0.1-85 wt. %, from 0.1-75 wt. %, from 1-75 wt. %, from 10-75 wt. %, from 10-50 wt. %, from 10-25 wt. %, from 25-50 wt. %, from 25-75 wt. %, from 50-75 wt. %, from 90-100 wt. %, from 95-100 wt. %, from 90-95 wt. %, from 75-100 wt. %, from 75-95 wt. %, from 30-45 wt. %, from 30-60 wt. %, from 40-75 wt. %, from 1-10 wt. %, from 1-5 wt. %, from 5-10 wt. %, from 5 -15 wt. %, or from 15-30 wt. %, relative to the total weight of the particles.

34. The composition according to claim 1, wherein the particles consist of haptoglobin, hemopexin, or transferrin.

35-36. (canceled)

37. The composition according to claim 1, wherein the particles consist of haptoglobin and hemopexin, haptoglobin and transferrin, or hemopexin and transferrin.

38-39. (canceled)

40. The composition according to claim 1, wherein the particles consist of haptoglobin, hemopexin, and transferrin.

41. The composition according to any of claim 1, wherein the composition comprises the particles in an amount from 1-5,000 μg/mL, from 1-2,500 μg/mL, from 1-1,000 μg/mL, from 1-500 μg/mL, from 1-250 μg/mL, from 1-100 μg/mL, from 1-50 μg/mL, from 1-25 μg/mL, from 1-10 μg/mL, from 5-25 μg/mL, from 10-50 μg/mL, from 25-50 μg/mL, from 25-75 μg/mL, from 25-100 μg/mL, from 50-100 μg/mL, from 75-100 μg/mL, from 75-125 μg/mL, from 50-150 μg/mL, from 100-250 μg/mL, from 250-500 μg/mL, from 500-1,000 μg/mL, from 1,000-2,500 μg/mL, from 1,000-5,000 μg/mL, or from 2,500-5,000 μg/mL, and/or wherein the particles have an average particle size from 1-500,000 nm, from 1-250,000 nm, 1-100,000 nm, from 1-50,000 nm, from 1-25,000 nm, from 1-10,000 nm, from 1-5,000 nm, from 1-1,000 nm, from 1-500 nm, from 1-250 nm, from 1-100 nm, from 1-50 nm, from 50-500 nm, from 50 -250 nm, from 100-500 nm, from 100-250 nm, from 250-500, from 500-750 nm, from 750-1,000 nm, from 500-5,000 nm, from 500-10,000 nm, from 1,000-5,000 nm, from 2,500-5,000 nm, from 1,000-10,000 nm, from 2,500-10,000 nm, from 5,000-10,000 nm, from 10,000-25,000 nm, from 10,000-50,000, from 25,000-50,000, from 25,000-75,000 nm, from 50,000-100,000, or from 50,000 -150,000 nm.

42. (canceled)

43. A method of treating or reducing ferroptosis, treating a disorder of the eye, treating an inflammatory disorder, or promoting healing in injured tissue in a subject in need thereof, comprising administering to the subject the composition according to claim 1.

44-54. (canceled)

55. A method of treating a disorder of the eye, comprising administering to a subject in need thereof a composition comprising a therapeutic protein, wherein the therapeutic protein comprises haptoglobin, hemopexin, transferrin, or a combination thereof.

56-97. (canceled)
